(19) United States
(12) Patent Application Publication
(10) Pub. No.: US 2015/0104380 A1
(43) Pub. Date: Apr. 16, 2015

(54) Biodegredable Carrier For Carrying Radioisotope And Kit Containing The Same

(71) Applicant: Institute of Nuclear Energy Research Atomic Energy Council, Executive Yuan, TAOYUAN COUNTY (TW)

(72) Inventors: LIANG-YU CHANG, TAOYUAN COUNTY (TW); TSAI-YUEH LUO, Taoyuan County (TW); MIN-SHENG PU, New Taipei City (TW); PING-FANG CHIANG, TAOYUAN COUNTY (TW); HUA-MIN WANG, TAOYUAN COUNTY (TW); YING-HSIA SHIH, Changhua County (TW)

(21) Appl. No.: 14/335,652

(22) Filed: Jul. 18, 2014

(30) Foreign Application Priority Data
Oct. 11, 2013 (TW) 102136780

(51) Int. Cl. A61K 51/06

(52) U.S. Cl. CPC A61K 51/06
USPC 424/1.25; 525/419

(57) ABSTRACT

The present invention relates to a biodegradable carrier for carrying a radioisotope, which is formed from at least one biodegradable polymer selected from the group consisting of poly(lactic-co-glycolic acid) (PLGA), poly(lactic acid) (PLA), poly(ε-caprolactone) (PCL), chitosan, poly(γ-glutamic acid) (PGA), and polyethylene glycol (PE) in which its hydroxyl group is substituted with an amino group and grafted with tetraazocyclododecanetetraacetic acid monosuccinimide ester (DOTA-NHS), in which nitrogen atoms contained in the DOTA-NHS are provided for coordinating with a radioisotope. The present invention also relates to a kit which includes a first container containing the biodegradable carrier for carrying a radioisotope according to the present invention and a second container containing a radioisotope.

### CROSS-REFERENCE TO RELATED APPLICATION

### Technical Field

The present disclosure relates to a biodegradable carrier for carrying a radioisotope, which is used for coordinating with a radioisotope to form a biodegradable carrier having a radioisotope and can be used for brachythearpy of tumor cells to transport the radioisotope to the inside or surrounding of a tumor cell lesion tissue, and uses a radioisotope to transmit radiation in a short distance to give high dose radiation for tumor and reduce the risk of damage to normal cells near a lesion.

### BACKGROUND

Currently, the liver cancer treatment includes surgical removal, vascular embolization, local ethanol injection, radiofrequency thermal cautery therapy, chemotherapy, radiotherapy, immunotherapy, etc., and age, tumor size, location of tumor growth and other physical conditions should be considered to decide the treatment manner. Although surgery can remove most lesions, according to clinical experience, only less than 15% of patients are suitable for surgery.

Regarding the traditional chemical embolization, it is confirmed that chemotherapy drugs by lipiodol infusion can stay in the tumor for only about 20 minutes to 2 hours, so the time of killing the tumor may be too short to achieve the expected effect, and systemic toxicity is also serious. Thus, radiological embolization is developed currently, which is to inject micro particles with radioactive rays such as yttrium into the liver tumor site by means of vascular embolization, to make the micro particles attached to the tumor, and kill cancer cells accurately by radiating high energy radiation. Because the radiation is pure β-rays, penetration is short, damage to surrounding normal liver cells can be reduced, and the family and medical personnel are less affected.

However, the currently used carrier for micro particles with radioactive rays takes glass and resin to carry radiopharmaceuticals, and the carrier is packaged and transported together with radioisotopes. Therefore, the transportation cost is high and the delivery process is difficult, and in use, the risk of inactivation of the isotope in the delivery process may occur to affect the curative effect.

In view of the above, the inventors have conducted extensive researches on the above problems, thereby accomplishing the present invention.

### SUMMARY

The present invention relates to a biodegradable carrier for carrying a radioisotope, which is used for coordinating with a radioisotope to form a biodegradable carrier having a radioisotope and can be used for brachythearpy of tumor cells to transport the radioisotope to the inside or surrounding of a tumor cell lesion tissue, which uses a radioisotope to transmit radiation in a short distance, and gives high dose radiation for tumor to reduce the risk of damage to normal cells near a lesion. The present invention mainly utilizes advantages of the brachythearpy, and coordinates with the biodegradable carrier for marking medical isotopes and coating chemotherapy drugs; the initial phase of therapy focuses on brachythearpy and uses radioactive rays to kill cancer cells and reduce the tumor size, and the biodegradable carrier is subsequently absorbed by the human body; such a local administration manner can decrease the administered dose to reduce patients' discomfort during the treatment and reduce side effects, and carrier materials can be absorbed by the human body and are different from glass and resin used in the current commercially available carrier, so use of biodegradable materials can avoid problems such as immune rejection generated after the patients use drugs.

The present invention is directed to a biodegradable carrier for carrying a radioisotope, which is formed from at least one biodegradable polymer selected from the group consisting of poly(lactic-co-glycolic acid) (PLGA), poly(lactic acid) (PLA), poly(ε-caprolactone) (PCL), chitosan, poly(γ-glutamic acid) (PGA), and polyethylene glycol (PE) in which its hydroxyl group is substituted with an amino group and grafted with tetraazocyclododecanetetraacetic acid monosuccinimide ester (DOTA-NHS), in which nitrogen atoms contained in the DOTA-NHS are provided for coordinating with a radioisotope.

In the biodegradable carrier for carrying a radioisotope according to the present invention, the radioisotope that can be used together therewith is a radioisotope that can emit β-rays, for example, at least one selected from the group consisting of Re-188, Re-186, Lu-177, Sm-153, I-131, In-111, Y-90 and Cu-64, and preferably at least one selected from In-111, Y-90 and Cu-64.

In the biodegradable carrier for carrying a radioisotope according to the present invention, weight average molecular weight of the biodegradable polymer is in a range of 17,000 to 105,000.

The biodegradable carrier for carrying a radioisotope according to the present invention is a micron-level particle. The micron-level particle as mentioned herein indicates a particle with a particle size in a range of 1 micron to 200 microns. In the biodegradable carrier for carrying a radioisotope according to the present invention, the biodegradable polymer is preferably any one of the following or a combination thereof: amine-modified PLGA, of which the number average molecular weight is in a range of 38000 to 54000, and a polymerization molar ratio of lactic acid to glycolic acid is 40:60 to 60:40; and amine-modified polyvinyl alcohol (PVA), of which the number average molecular weight is in a range of 20000 to 30000 and the hydrolysis rate is 70 to 90%.

Before the biodegradable carrier for carrying a radioisotope according to the present invention is to be used for treating tumor cells, that is, to be used for brachythearpy to transport a radioisotope to the inside or surrounding of a tumor cell lesion tissue, it is only necessary to dissolve or disperse the biodegradable carrier according to the present invention in a phosphate buffer, add a radioisotope solution with a desired amount of radioactivity, and mix them for 30 to 180 minutes at a temperature of 20 to 45° C., and the biodegradable carrier grafted with a radioisotope can be obtained.

In the biodegradable carrier for carrying a radioisotope according to the present invention, the carrier can be internally coated with active pharmaceutical ingredients, and the active ingredients are not specifically limited, and appropriate active ingredients may be selected according to therapeutic purposes. Accordingly, after the biodegradable carrier coated with drugs according to the present invention coordinates with the radioisotope, it enables the drugs and the radioisotope to have a synergistic effect, and the frequency of administration can be reduced, thereby reducing patients' discomfort or anxiety.

A method for manufacturing a biodegradable carrier for carrying a radioisotope according to the present invention, including the following steps of: (1) substituting a hydroxyl group of at least one biodegradable polymer selected from the group consisting of poly(lactic-co-glycolic acid) (PLGA), poly(lactic acid) (PLA), poly(ε-caprolactone) (PCL), chitosan, poly(γ-glutamic acid) (PGA), and polyethylene glycol (PE) with an amino group of a compound containing amine functional groups, for example, N-tertiary-butoxycarbonyl glycine, N-(tertiary-butoxycarbonyl)-L-alanine in the presence of alkali selected from, for example, dicyclohexyl carbodiimide (DCC), N,N′-diisopropyl carbodiimide (DIC) or 1-ethyl-(3-dimethylaminopropyl) carbodiimide (EDC) and dimethylaminopyridine (DMAP), to obtain an amine-containing biodegradable carrier, in which the usage amount of the compound containing amine functional groups is 3 to 5 times the molar amount relative to 1 mole of the hydroxyl group of the biodegradable polymer, and the usage amount of the alkali is 3 to 5 times the molar amount relative to 1 mole of the hydroxyl group of the biodegradable polymer; and (2) then reacting the amine-containing biodegradable carrier with a chelating agent selected from tetraazocyclododecanetetraacetic acid (DOTA) or diethylene triamine pentaacetic acid (DTPA), and succinimide selected from N-hydroxysuccinimide (NHS) or sulfo-N-hydroxy-succinimide (sulf-NHS) in the presence of alkali selected from, for example, dicyclohexyl carbodiimide (DCC), N,N′-diisopropyl carbodiimide (DIC) or 1-ethyl-(3-dimethylaminopropyl) carbodiimide (EDC), in which the usage amount of the chelating agent is 3 to 5 times the molar amount relative to 1 mole of the hydroxyl group of the biodegradable polymer, the usage amount of the succinimide is 2 to 5 times the molar amount relative to 1 mole of the hydroxyl group of the biodegradable polymer, and the usage amount of the alkali is 2 to 5 times the molar amount relative to 1 mole of the hydroxyl group of the biodegradable polymer, so as to obtain the biodegradable carrier for carrying a radioisotope.

The present invention also relates to a kit, including a first container containing the biodegradable carrier for carrying a radioisotope; and a second container containing a radioisotope.

In the kit according to the present invention, a ratio of the biodegradable carrier to the radioisotope is decided by a desired amount of radioisotope, which cannot be generalized. Usually per gram of biodegradable carrier can be mixed with a radioisotope with 100 mCi to 150 mCi (3700 MBq to 5550 MBq) radioactivity.

In the kit according to the present invention, the radioisotope used in the second container is a radioisotope that can emit β-rays, for example, at least one selected from the group consisting of Re-188, Re-186, Lu-177, Sm-153, I-131, In-111, Y-90 and Cu-64, and preferably at least one selected from In-111, Y-90 and Cu-64.

In the kit according to the present invention, the biodegradable polymer in the first container is preferably any one of the following or a combination thereof: amine-modified PLGA, of which the number average molecular weight is in a range of 38000 to 54000, and a polymerization molar ratio of lactic acid to glycolic acid is 40:60 to 60:40; and amine-modified PVA, of which the number average molecular weight is in a range of 20000 to 30000 and the hydrolysis rate is 70 to 90%.

Shortly before using the kit according to the present invention, a phosphate buffer or physiological saline is added to the first container to disperse the biodegradable carrier to obtain a solution with a pH in a range of 7.0 to 8.0, and then the radioisotope is added in the second container therein and the two are mixed for 30 to 180 minutes at a temperature of 20 to 45° C., and the biodegradable carrier grafted with a radioisotope can be obtained; moreover, the kit is used for brachythearpy of tumor cells to transport the radioisotope to the inside or surrounding of a tumor cell lesion tissue. The radioisotope is used to transmit radiation in a short distance to give high dose radiation for tumor and reduce the risk of damage to normal cells near a lesion.

The amount of the phosphate buffer or physiological saline for dispersing the biodegradable carrier is not specifically limited, as long as the biodegradable carrier can be dispersed; however, in order that the dispersion is not too thin, 5 to 15 ml, preferably 8 to 12 ml, of physiological saline or phosphate buffer is used relative to per gram of biodegradable carrier preferably.

When a medium for dispersing the biodegradable carrier is phosphate buffer, after the biodegradable carrier in the first container is reacted with the radioisotope in the second container to obtain a solution of the biodegradable carrier grafted with a radioisotope, it is desired to further remove the phosphate buffer by centrifugation and then inject it into an animal body especially a human body after dispersing it with the physiological saline suitable for injection to an animal body especially a human body.

### BRIEF DESCRIPTION OF DRAWINGS

FIG. 1 shows a single-photo emission computed tomography (SPECT) image after an isotopically-marked DOTA-NHS-PLGA micron carrier is injected into a rat according to the present invention.

### DETAILED DESCRIPTION OF DISCLOSED EMBODIMENTS

A biodegradable carrier for carrying a radioisotope according to the present invention is formed from at least one biodegradable polymer selected from the group consisting of poly(lactic-co-glycolic acid) (PLGA), poly(lactic acid) (PLA), poly(ε-caprolactone) (PCL), chitosan, poly(γ-glutamic acid) (PGA), and polyethylene glycol (PE) in which its hydroxyl group is substituted with an amino group and grafted with tetraazocyclododecanetetraacetic acid monosuccinimide ester (DOTA-NHS), in which nitrogen atoms contained in the DOTA-NHS are provided for coordinating with a radioisotope.

In the biodegradable carrier for carrying a radioisotope according to the present invention, weight average molecular weight of the biodegradable polymer is in a range of 17,000 to 105,000.

In the biodegradable carrier for carrying a radioisotope according to the present invention, the biodegradable polymer used therein may be synthesized with a method known in the art or may be commercially obtained, for example, PLGA with CAS No. 26780-50-7 purchased from Sigma-Aldrich may be used, of which the number average molecular weight is in a range of 38000 to 54000, and a polymerization molar ratio of lactic acid to glycolic acid is 50:50; or PVA with CAS No. 9002-89-5 purchased from ACROS may be used, of which the number average molecular weight is in a range of 20000 to 30000 and the hydrolysis rate is 88%.

In the biodegradable carrier for carrying a radioisotope according to the present invention, an appropriate radioisotope can be selected for the radioisotope that can be used together therewith according to a target tumor to be treated, which is not specifically limited. However, according to the radioisotope used in the current tumor therapy, a radioisotope that can emit β-rays can be used, for example, selected from the group consisting of Re-188, Re-186, Lu-177, Sm-153, I-131, In-111, Y-90 and Cu-64, and preferably In-111, Y-90 or Cu-64.

As the biodegradable carrier for carrying a radioisotope according to the present invention is grafted with DOTA-NHS for coordinating with the radioisotope, in medical use, the biodegradable carrier for carrying a radioisotope and the radioisotope can be separated, and they are mixed shortly before use, so as to obtain a DOTA-NHS-PLGA micron carrier marked with a radioisotope. Thus, the present invention also includes a kit, including a first container containing the biodegradable carrier for carrying a radioisotope according to the present invention, and a second container containing a radioisotope. Shortly before use, contents in the two containers are mixed, to obtain a biodegradable carrier marked with a radioisotope.

### EMBODIMENTS

The present invention is specifically described with the following embodiments; however, the embodiments are only illustrative but are not intended to limit the scope of the present invention.

### (1) Surface Modification of a Biodegradable Carrier

This embodiment uses PLGA as a modified material, of which the molecular weight is 38000 to 54000.500 mg of a PLGA polymer material was taken, and 25 mg of N,N′-dicyclohexyl carbodiimide (DCC) and 100 mg of 4-(dimethylamino) pyridine (DMAP) were added for reaction, and about 200 mg of N-tertiary-butoxycarbonyl glycine was added in which a —NH₂ functional group served as a source of surface modification. The mixture was dissolved in 10 ml of methylene chloride and placed in a refrigerator at 4° C. for 24-hour reaction, a polymer mass was extracted with 10 ml of methanol and waste liquid was removed, 10 ml of trifluoroacetic acid and 10 ml of a dichloromethane solution were added to dissolve the polymer mass at room temperature (25° C.) for 3-hour reaction, extraction and purification were performed with more than 20 ml of methanol, and the product was placed in a freeze dryer to remove an excess organic phase upon removal of the waste liquid. 450 mg of a PLGA material (PLGA-NH₂) surface-modified to a NH₂ group was. The reaction process is as follows:

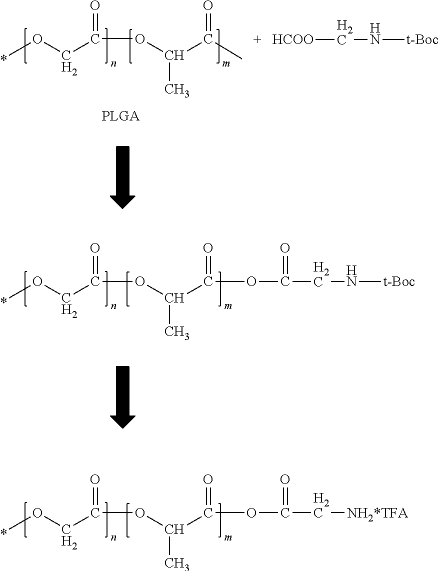

### (2) Preparation of a Micron Ball of an Amine-Modified Biodegradable Carrier

500 mg of the PLGA-NH₂ material obtained in the step (1) was dissolved with 1 ml of dichloromethane (the PLGA concentration was in a range of 0.5 wt % to 10 wt %), and 0.5 wt % to 3 wt % of a PVA solution was prepared; the dichloromethane solution containing a PLGA-NH₂ carrier was dropped slowly into the PVA solution with a glass dropper (the PVA in this step has functions of a biodegradable carrier and a surfactant, to assist in forming the PLGA carrier), the mixed liquid was stirred with a homogenizer after dripping, and particles were prepared by means of emulsion. Next, filter membranes with pore sizes of 25 μm and 47 μm were used to filter out micron particles whose particle size is between 25±10 μm to 47±10 μm respectively, and vacuum concentration was performed to remove the organic phase, dry micron particles were collected in a freeze-drying manner to obtain PLGA-NH₂ micron particles, and the particle shaping result was with an SEM.

### (3) Preparation of a Biodegradable Micron Carrier Grafted with DOTA-NHS

A coupling reaction between the PLGA-NH₂ micron particles prepared in the step (2) and DOTA-NHS was performed in the following manner. First 15 mg of DOTA, 30 mg of EDC and 40 mg of sulf-NHS were dissolved into 2 ml of pure water, a pH value of the solution was adjusted to 7.5 with a Na₂HPO₄ solution, the product was stirred at room temperature, 100 mg of the PLGA-NH₂ micron particles prepared in the step (2) were added, reaction was carried out for 24 hours at a temperature of 4° C., unreacted DOTA was removed with pure water and pumping filtration equipment, and the purified solution was freeze dried to obtain 380 mg of a biodegradable micron carrier grafted with DOTA-NHS (DOTA-NHS-PLGA micron carrier).

### (4) Preparation of a DOTA-NHS-PLGA Micron Carrier Marked with a Radioisotope

100 mg of the DOTA-NHS-PLGA micron carrier obtained in the step (3) was dissolved in 1 ml of the phosphate buffer, a ¹¹¹InCl₃-containing 0.05N HCl solution (In content calculated by activity is about 1 mCi (3.7 MBq)) was added, and reaction was carried out for 60 minutes at 37° C. to obtain 100 mg of a DOTA-NHS-PLGA micron carrier marked with a radioisotope (¹¹¹In-DOTA-NHS-PLGA micron carrier) (the yield is 100%, and the radioactivity of the sample is between 0.9 mCi to 1 mCi).

### (5) Qualitative and Quantitative Analysis on the DOTA-NHS-PLGA Micron Carrier Marked with a Radioisotope

Radiochemical purity analysis on a product was performed with an ITLC-SG/NS system coordinating with a radioactive thin-layer chromatograph (Radio-TLC). Free ¹¹¹InCl₃ without coordination may move to the position of a solvent point (Rf=1), while the ¹¹¹In-DOTA-NHS-PLGA micron carrier completing the coordination may stay at the origin (Rf=0.0), so as to analyze the flag rate and radiochemical purity of the product.

The radiochemical purity was measured as follows: Isotope TLC (ITLC) was used to measure fixed-direction radiation intensity to obtain a binding rate between an isotope and a PLGA carrier and stability of the sample, for example, the PLGA carrier having unconnected free isotopes may move with a flowing direction and has radiation intensity signal representation.

The reaction process of the steps (3) and (4) is as follows.

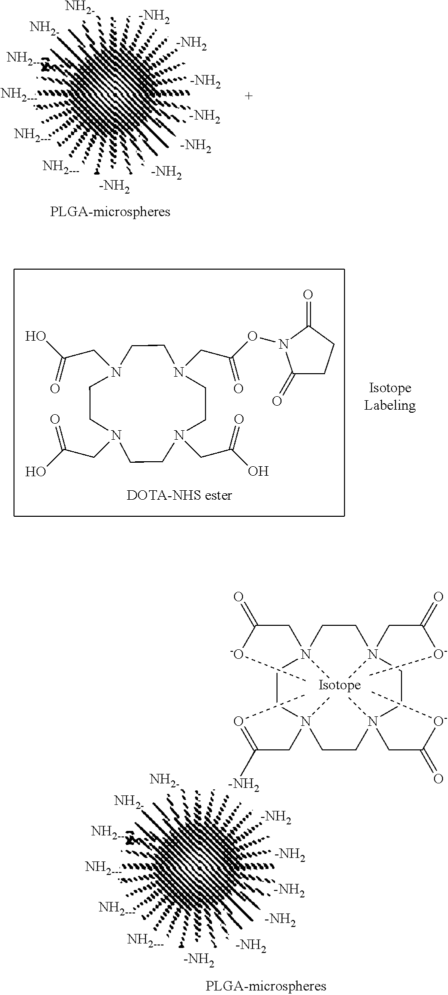

### EXPERIMENTAL EXAMPLE

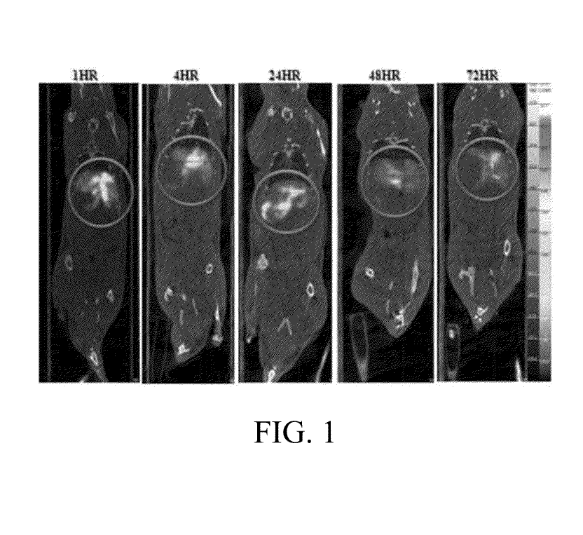

0.15 ml of physiological saline in 15 mg of the DOTA-NHS-PLGA micron carrier marked with a radioisotope prepared in the above embodiments was used as a liver embolization agent, and was injected into a rat (weighed about 250 g to 300 g) with a syringe, and then SPECT was performed to obtain an image as shown in FIG. 1. It can be known from FIG. 1 that the DOTA-NHS-PLGA micron carriers marked with a radioisotope according to the present invention stay at the liver part of the rat and do not escape to other organs, and thus the DOTA-NHS-PLGA micron carriers marked with a radioisotope according to the present invention suitably serve as liver embolization agents.

In view of the above, as the biodegradable carrier for carrying a radioisotope according to the present invention is grafted with DOTA-NHS for coordinating with the radioisotope, in medical use, the biodegradable carrier for carrying a radioisotope and the radioisotope can be separated, and they are mixed shortly before use, so as to obtain a DOTA-NHS-PLGA micron carrier marked with a radioisotope. Therefore, it is helpful to transport drugs and reduce freight as well as avoid the risk of activity decay of radioisotopes, so that sale of medicines is more flexible and the cost can be reduced.

In addition, as the biodegradable carrier for carrying a radioisotope according to the present invention and the radioisotope are separated, products can be provided in a form of a kit, and different radioisotopes are selected for different tumors according to parts to be treated or usage. Moreover, the biodegradable carrier for carrying a radioisotope according to the present invention, in addition to coordinating with the radioisotope, can coat required drugs internally, and most suitable chemotherapy drugs and medical isotopes can be designed to increase diversity of tumor therapy.

As the biodegradable carrier for carrying a radioisotope according to the present invention is biodegradable, when used in vivo, the biodegradable carrier is absorbed by the living body with the lapse of time, which can avoid problems such as immune rejection generated after the patients use drugs. It also can avoid load of drug accumulation on the human body when radioisotope injection is performed on a patient multiple times due to the needs of therapy.

Accordingly, after the biodegradable carrier coated with drugs according to the present invention coordinates with the radioisotope, it enables the drugs and the radioisotope to have a synergistic effect, and the frequency of administration can be reduced, thereby reducing patients' discomfort or anxiety. Hence, the biodegradable carrier for carrying a radioisotope according to the present invention has availability in medical use.

What is claimed is:

1. A biodegradable carrier for carrying a radioisotope, which is formed from at least one biodegradable polymer selected from the group consisting of poly(lactic-co-glycolic acid) (PLGA), poly(lactic acid) (PLA), poly(ε-caprolactone) (PCL), chitosan, poly(γ-glutamic acid) (PGA), and polyethylene glycol (PE) in which its hydroxyl group is substituted with an amino group and grafted with tetraazocyclododecanetetraacetic acid monosuccinimide ester (DOTA-NHS).

2. The biodegradable carrier for carrying a radioisotope according to claim 1, wherein weight average molecular weight of the biodegradable polymer is in a range of 17,000 to 105,000.

3. The biodegradable carrier for carrying a radioisotope according to claim 1, wherein the biodegradable polymer is PLGA, of which the number average molecular weight is in a range of 38000 to 54000, and a polymerization molar ratio of lactic acid to glycolic acid is 40:60 to 60:40.

4. The biodegradable carrier for carrying a radioisotope according to claim 1, wherein the biodegradable polymer is polyvinyl alcohol (PVA), of which the number average molecular weight is in a range of 20000 to 30000 and the hydrolysis rate is 70 to 90%.

5. The biodegradable carrier for carrying a radioisotope according to claim 1, wherein the biodegradable carrier is a micron-level particle.

6. The biodegradable carrier for carrying a radioisotope according to claim 1, wherein the biodegradable carrier for carrying a radioisotope is formed by utilizing nitrogen atoms contained in the DOTA-NHS to coordinate with a radioisotope.

7. The biodegradable carrier for carrying a radioisotope according to claim 6, wherein the radioisotope is at least one selected from the group consisting of Re (rhenium)-188, Re-186, Lu (lutetium)-177, Sm (samarium)-153, I (iodine)-131, In (indium)l11, Y (yttrium)-90 and Cu (copper)-64.

8. The biodegradable carrier for carrying a radioisotope according to claim 7, wherein the radioisotope is at least one selected from In-111, Y-90 and Cu-64.

9. The biodegradable carrier for carrying a radioisotope according to claim 1, wherein the biodegradable carrier is further internally coated with active pharmaceutical ingredients.

10. A kit, comprising a first container containing the biodegradable carrier for carrying a radioisotope according to claim 1, and a second container containing a radioisotope.

11. The kit according to claim 10, wherein the biodegradable polymer in the first container is poly(lactic-co-glycolic acid) (PLGA), of which the number average molecular weight is in a range of 38000 to 54000, and a polymerization molar ratio of lactic acid to glycolic acid is 40:60 to 60:40.

12. The kit according to claim 10, wherein the biodegradable polymer in the first container is polyvinyl alcohol (PVA), of which the number average molecular weight is in a range of 20000 to 30000 and the hydrolysis rate is 70 to 90%.

13. The kit according to claim 10, wherein the radioisotope used in the second container is at least one selected from the group consisting of Re (rhenium)-188, Re-186, Lu (lutetium)-177, Sm (samarium)-153, I (iodine)-131, In (indium)-111, Y (yttrium)-90 and Cu (copper)-64.

14. The kit according to claim 10, wherein the radioisotope used in the second container is at least one selected from In-111, Y-90 and Cu-64.

15. The kit according to claim 10, wherein the biodegradable carrier for carrying a radioisotope in the first container is further internally coated with active pharmaceutical ingredients.
